(19) United States
(12) Patent Application Publication
(10) Pub. No.: US 2012/0298101 A1
(43) Pub. Date: Nov. 29, 2012

(54) Magnet Aided Intubation Systems, Kits, and Methods

(76) Inventors: Alexander H. Cooper, Savannah, GA (US); Elizabeth A. Carrigan, Atlanta, GA (US); Shawna M. Hagen, Red Bank, NJ (US); William J. Thompson, Cornelia, GA (US)

(21) Appl. No.: 13/415,707

(22) Filed: Mar. 8, 2012

Related U.S. Application Data
(60) Provisional application No. 61/450,400, filed on Mar. 8, 2011.

(51) Int. Cl. A61M 16/04

(52) U.S. Cl. 128/200.26

(57) ABSTRACT

Endotracheal intubation systems, methods, and kits are provided. The system may include an endotracheal tube assembly which includes an endotracheal tube having a proximal end and a distal end; a wire having a proximal end and a distal end, wherein the wire is configured to be removably inserted into the endotracheal tube through the proximal end of the endotracheal tube; an internal magnetic member fixed at the distal end of the wire; and an end cap attached to the proximal end of the wire and removably securable to the proximal end of the endotracheal tube, wherein at least a portion of the internal magnetic member is disposed within the distal end of the endotracheal tube at a fixed position when the end cap is secured to the proximal end of the endotracheal tube; and an external magnetic member positionable on the exterior of a patient.

### CROSS-REFERENCE TO RELATED APPLICATIONS

This application claims priority to U.S. Provisional Patent Application No. 61/450,400, filed on Mar. 8, 2011, the disclosure of which is incorporated herein by reference.

### BACKGROUND

This disclosure is generally related to medical systems and methods for insertion of an endotracheal tube into a patient's airway.

Conventional intubation devices have provided various guide mechanisms to direct an intubation tube into the trachea of a patient. A laryngoscope is typically required to facilitate viewing the pathway to enable the intubation procedure. Conventional intubation devices are inserted by first inserting a guide device, such as a guide wire, insertion sheath, and/or guide cylinder into the trachea. An intubation tube then is connected to the guide wire or inserted through the sheath or guide cylinder for intubation. The intubation process typically requires a trained attendant to manipulate a guide wire or tube into the trachea by rotating, wiggling, and turning the guide device until the tube is inserted into the trachea. The intubation process is often slow and difficult and, if the procedure is not completed quickly and properly, the patient may suffer injury from a lack of oxygen.

The use of magnets to facilitate intubation are known, such as disclosed in U.S. Pat. No. 6,672,308 to Gaspari. However, that system requires the use of a laryngoscope, which may break the patient's teeth and is a vector for pathogens. In addition, the Gaspari system uses a “neck brace” for placement of the exterior guide magnet, which may prevent the medical professional from receiving useful tactile feedback from the interaction of the magnets to insure tracheal placement of the tube.

In another prior art system, the endotracheal tube has a magnetic tip. However, this presents another difficulty in that a magnetic object is internalized within a patient for the duration of intubation, thereby necessitating reintubation if any magnetically-based medical or diagnostic procedures (e.g., NMRI) are required on the patient, since the magnet cannot remain in the patient during such procedures.

Thus, there remains a need to provide improved intubation device designs and methods of intubation that are rapid, reliable, inexpensive, and easy to use, preferably without the need for use of a laryngoscope.

### SUMMARY

Thus, there remains a need to provide improved intubation device designs and methods of intubation that are rapid, reliable, inexpensive, and easy to use, preferably without the need for use of a laryngoscope.

In one aspect, an endotracheal intubation system is provided that includes an endotracheal tube assembly which comprises an endotracheal tube having a proximal end and a distal end; a wire having a proximal end and a distal end, wherein the wire is configured to be removably inserted into the endotracheal tube through the proximal end of the endotracheal tube; an internal magnetic member fixed at the distal end of the wire; and an end cap attached to the proximal end of the wire and removably securable to the proximal end of the endotracheal tube, wherein at least a portion of the internal magnetic member is disposed within the distal end of the endotracheal tube at a fixed position when the end cap is secured to the proximal end of the endotracheal tube; and an external magnetic member positionable on the exterior of a patient. The end cap may further include a carbon dioxide detector. The external magnetic member may comprise an outer shell, a magnet disposed therein, and an adjustable positioning member for selectively securing the magnet in two or more positions within the outer shell.

In another aspect, a kit of parts is provided that includes a first part comprising a wire having a proximal end and a distal end, an internal magnetic member fixed at the distal end of the wire, and an end cap attached to the proximal end of the wire, wherein the end cap is removably securable to a proximal end of an endotracheal tube with the wire removably inserted into the endotracheal tube and at least a portion of the internal magnetic member disposed within the distal end of the endotracheal tube; and a second part comprising external magnetic member positionable on the exterior of a patient, wherein the first and second parts are configured to cooperate to magnetically guide the endotracheal tube into a patient's trachea.

In another aspect, a method is provided for intubating a patient. The method may include positioning an external magnetic member adjacent to a cricothyroid cartilage of the patient; inserting though the patient's mouth and into the patient's trachea a distal end of an endotracheal tube assembly which comprises (a) an endotracheal tube comprising a flexible, hollow tube having a distal end and a proximal end, (b) a wire having a proximal end and a distal end, (c) an internal magnetic member fixed at the distal end of the wire, and (d) an end cap fixed to the proximal end of the wire and removably secured to the proximal end of the endotracheal tube, wherein at least a portion of the internal magnetic member is disposed within the distal end of the endotracheal tube; and then unsecuring the end cap from the proximal end of the endotracheal tube and withdrawing the wire and internal magnetic member from the endotracheal tube through the proximal end of the endotracheal tube, while maintaining the endotracheal tube within the patient's trachea.

### BRIEF DESCRIPTION OF THE DRAWINGS

The present disclosure may be better understood with reference to the following figures.

FIG. 1 is an exploded perspective view of an endotracheal tube intubation system in accordance with an embodiment of the present description.

FIG. 2 contains two perspective views of an end cap of the endotracheal tube intubation system in accordance with an embodiment of the present description.

FIG. 3 is a schematic illustration of an external magnetic member and an optional harness in accordance with embodiments of the present description.

FIGS. 4A-4D are a side elevation view (A), top perspective view (B), bottom perspective view (C), and top elevation view (D) of an end cap having a carbon dioxide detector in accordance with an embodiment of the present description.

FIG. 5 is a plan view of an endotracheal tube intubation system in its assembled configuration for tracheal insertion in accordance with an embodiment of the present description.

FIGS. 6A and 6B are perspective views of an unassembled (A) and assembled (B) external magnetic member in accordance with an embodiment of the present description.

FIGS. 7A and 7B are a plan view and an exploded view of an internal magnetic member in accordance with an embodiment of the present description.

### DETAILED DESCRIPTION

Improved intubation systems have been developed for proper placement of endotracheal tubes, without requiring use of traditional visual assisting means, such as a laryngoscope. In embodiments, the intubation systems provide a number of advantages. When the endotracheal tube system is assembled and configured for deployment, an internal magnetic member advantageously is positioned within the distal end of the endotracheal tube and an end cap of the assembly secured to the proximal end of the endotracheal tube provides that intubation is easily achieved without the internal magnet being pushed back out as the endotracheal tube is advanced into the patient's airway. In a preferred embodiment, the system uses a shape memory (spring back) wire to position the internal magnetic, which during testing was found to make the intubation process easier.

In one embodiment, the system includes a manually held exterior magnet, which was found to provide the user tactile feedback when the interior and exterior magnets interact when the tube is placed into the trachea, while nothing is felt when misdirecting the tube into the oesophagus, thereby providing placement confirmation.

### A. Endotracheal Tube Assembly

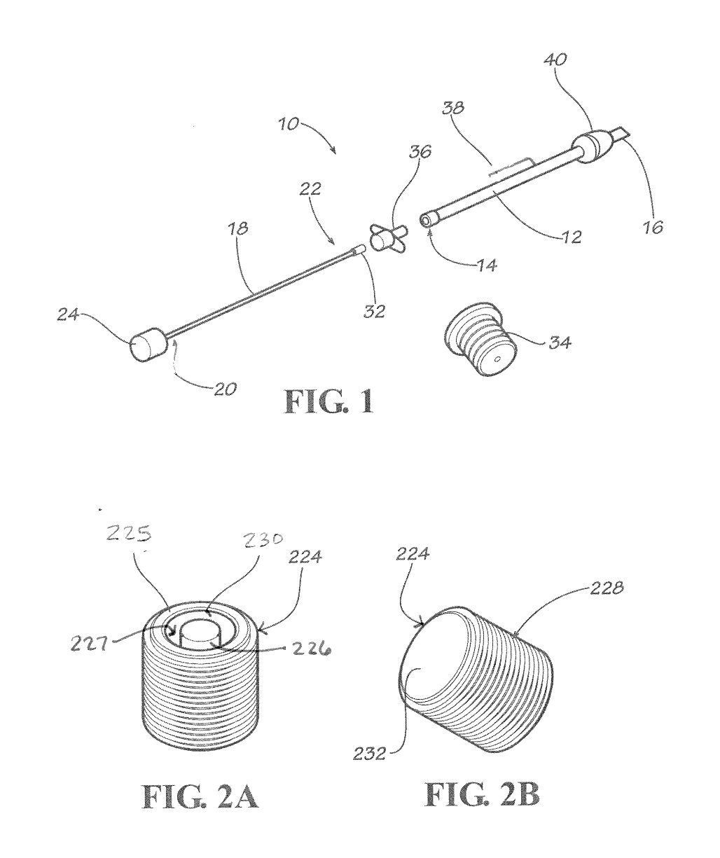

FIG. 1 shows an embodiment of an endotracheal tube assembly 10 and external magnet member 34. The endotracheal tube assembly 10 comprises a substantially hollow and flexible endotracheal tube 12 having a proximal end 14 and a distal end 16. The distal end 16 of the endotracheal tube 12 is configured for intubation, or insertion, of the endotracheal tube through the patient's mouth or nose and past the vocal cords into the trachea. The proximal end 14 of the endotracheal tube 12 is configured to remain outside of the patient, for example, for connection to an external assisted respiration device. In embodiments, the endotracheal tube 12 may be a standard, sterilized and commercially-available endotracheal tube 12 known and commonly used for intubation by persons of ordinary skill in the art.

The endotracheal assembly 10 further comprises a wire 18 having a proximal end 20 and a distal end 22. An internal magnetic member 32 is attached to the wire 18 at its distal end 22. An end cap 24 is attached to the wire 18 at its proximal end 20 and also may be configured to be removably attached to the proximal end 14 of the endotracheal tube 12. When the endotracheal tube assembly 10 is configured for deployment (insertion), the end cap 24 is removably attached to the proximal end 14 of the endotracheal tube 12 such that at least a portion of the internal magnetic member 32 may be positioned within the distal end 16 of the endotracheal tube 12, or may extend beyond the distal end 16 of the endotracheal tube 12 as described below.

In embodiments, the endotracheal tube assembly 10 may further comprise a resuscitator connector 36 disposed at the proximal end 14 of the endotracheal tube 12 and configured to securely and removably connect the proximal end 14 of the endotracheal tube 12 to the end cap 24, thereby securing the flexible wire 18 within the endotracheal tube 12. The resuscitator connector 36 may be a standard, sterilized and commercially-available resuscitator connector 36 that is commonly used with existing intubation tubes.

The end cap 24 advantageously positions and secures the wire 18 and the internal magnetic member 32 to improve in the intubation process by preventing the wire from pushing backwards out of the proximal end 14 of the endotracheal tube 12 as the tube 12 is advanced into the patient. After intubation is successfully completed, the wire 18 together with the internal magnetic member 32 may be removed from the endotracheal tube 12 using the end cap 24 to conveniently grip and remove the wire 18 and connected internal magnetic member 32.

In embodiments, the endotracheal tube assembly 10 may further comprise an inflatable cuff 40 disposed at or near the distal end 16 of the endotracheal tube 12. After intubation, the cuff 40 may be inflated with air, in a manner similar to the inflation of a balloon, to seal the patient's airway around the endotracheal tube 12. The inflatable cuff 40 may be inflated or deflated by introducing air through an air delivery line 38 in fluid communication with the inflatable cuff 40. A one-way valve (not shown) may be disposed between the air delivery line 38 and the inflatable cuff 40 or at a distal end of the air delivery line 38 to prevent air from leaking from the inflatable cuff.

The endotracheal tube assembly 10 may be made from any biocompatible materials having properties suitable for the intended functions of the particular elements. Various embodiments of the endotracheal tube assembly and its elements are described in more detail below.

The Endotracheal Tube

The endotracheal tube 12 desirably has flexible tube walls that bend both laterally and longitudinally when inserted into a patient. The walls of the endotracheal tube 12, however, should be sufficiently rigid to prevent internal collapse of the walls of the endotracheal tube 12 during its use. For example, the endotracheal tube may be composed of plastics, biopolymers and/or thermoplastic polymers, non-limiting examples of which include polyvinyl chloride, polyethylene, silicone, polypropylene, other flexible plastic materials, and combinations thereof.

The endotracheal tube 12 has a length that is sufficient both to connect the tube to an external assisted respiration device and to position the endotracheal tube 12 properly within the trachea. For example, the length of the endotracheal tube may be from about 7.5 cm to about 24 cm, from about 14 cm to about 21 cm, or from about 21 cm to about 24 cm. The endotracheal tube also desirably has an inner diameter sufficiently large to introduce a desired airflow into the patient's lungs and an outer diameter sufficiently narrow to comfortably insert the endotracheal tube assembly into the patient's trachea. For example, in some embodiments the endotracheal tube 12 may have an inner diameter from of about 2.5 mm to about 10 mm, from about 3.5 mm to about 8 mm, or from about 8 mm to about 9.5 mm, and an outer diameter from about 3.4 mm to about 13 mm, from about 4.5 mm to about 10.5 mm, or from about 11 mm to about 13 mm. Those skilled in the art will appreciate, however, that the particular dimensions of the endotracheal tube being used in embodiments of the intubation systems and methods provided herein necessarily will vary with the size of the patient.

The Wire Assembly

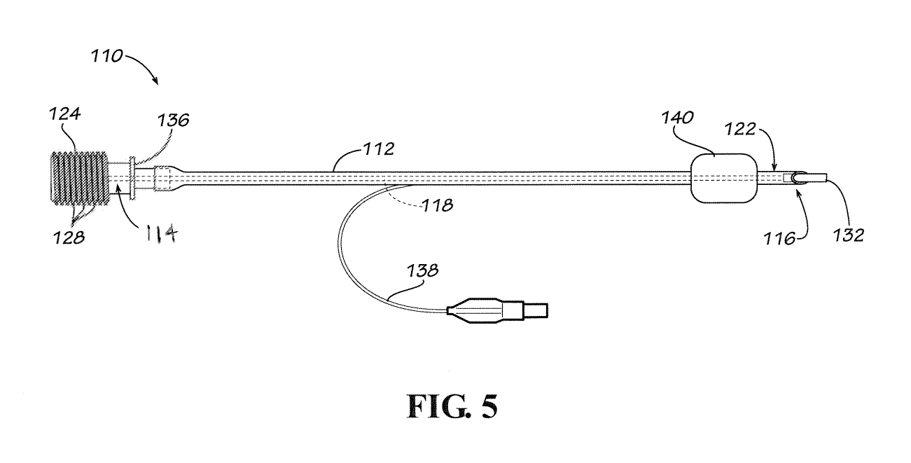

The wire 18 is configured to be inserted and removed (“removably inserted”) from the endotracheal tube 12 as needed before, during, or after use. For example, the wire 18 may be inserted into the endotracheal tube 12 by introducing the distal end 22 of the wire 18 into the endotracheal tube 12 through the proximal end 14 of the endotracheal tube 12. The wire 18 also is configured to be removed from the endotracheal tube 12 through the proximal end 16 of the endotracheal tube 12. Thus, the wire 18 may be inserted into and removed from the endotracheal tube 12 any number of times. Although the wire 18 may be inserted into the endotracheal tube 12 at any time, it is preferred to prepare the endotracheal tube assembly 10 such that it is configured for deployment into the patient (e.g., as illustrated in FIG. 5). The wire 18 desirably is freely rotatable within the endotracheal tube 12 and has an outer diameter sufficiently less than the inner diameter of the endotracheal tube 12 to allow for the passage of fluids in the space between the wire 18 and the endotracheal tube 12, thereby reducing and/or altogether avoiding the risk the fluids becoming occluded by the wire 18.

The wire 18 may comprise any biocompatible material that has the desired lateral flexibility and longitudinal (axial) stiffness for use in the intubation process. The thickness of the material(s) should render the wire laterally flexible and substantially inextensible. The diameter of the wire may be constant or varied along its length. For example, the wire may taper toward the distal end to provide relatively greater flexibility in that portion of the wire.

For example, the wire 18 may comprise a metal selected from a nickel-titanium alloy (e.g., Nitinol), a titanium-molybdenum alloy (e.g., Flexium), aluminum, steel, titanium, zinc, bronze, or a combination thereof. Other metals or alloys also may be used.

In other embodiments, the wire 18 may be formed of a polymer material. Examples of suitable polymer materials of construction include, but are not limited to, polypropylene, poly vinyl chloride, silicone, and polyethylene (e.g., ultra high molecular weight polyethylene). The polymer wire may be of solid construction or in the form of an annular tube.

In some embodiments, the wire 18 comprises a coating material, such as to impart biocompatibility, to lower the coefficient of friction between the wire and the endotracheal tube, and/or to increase the stiffness of the wire. Non-limiting examples of suitable coating materials include biopolymers and thermoplastic polymers. For example, the coating material may be selected from polytetrafluoroethylene (PTFE), polypropylene, polybutene, parylene, polvinyl chloride, or a combination thereof.

The wire 18 may be mononolithic or composed of multiple sections. Each of the one or more sections may comprise the same or a different materials. The wire may be a composite or an alloy. The materials of wire may be composed of a mixture or blend of two or more flexible materials.

In a preferred embodiment, the wire is or includes a spring-back (i.e., elastic) wire to provide both the desired lateral flexibility and axial rigidity. In a particular embodiment, the wire is tempered stainless steel, e.g., spring tempered, or tempered to impart a spring-back functionality to the wire. This allows for lateral deflection when subjected to a compressive force (i.e., is under load) but has a “shape memory” to cause it to return to its original shape/position when the compressive load is removed. The wire comprises may be formed from a superelastic alloy or shape memory material and “programmed” into a desired curved shape via a heat treatment process.

In one embodiment, the wire comprises 0.033″ diameter type 304 stainless steel wire with a tensile strength of 1.9-2.2 GPa (according to ASTM A313). In a preferred embodiment, the wire has a radius of curvature from about 4 inches to about 6 inches in an unloaded state.

The wire 18 further comprises an internal magnetic member 32 attached to the distal end 22 of the wire 18. As used herein, the term “internal magnetic member” means that the magnetic member is configured for use inside of a patient and the magnetic member comprises a magnetic material that is magnetically attracted to the external magnetic member. For example, in some embodiments the internal magnetic member comprise a magnetic material, a ferromagnetic material, a rare-earth magnet, or a combination thereof. In embodiments, the internal magnetic member may be a diametrically magnetized magnet or an axially magnetized magnet.

In a preferred embodiment, the internal magnet member comprises an elongated cylindrical magnet, which is diametrically magnetized. In a preferred embodiment, the axis of the cylindrical internal magnet member is oriented parallel to, and optionally coextensive with, the axis of the wire to which it is connected. This embodiment was found to function superior to an axially magnetized magnet due to the directionality of the poles, as magnets attract each other when the fields are aligned in the same direction. Generally, diametric magnets allow for pole alignment when in the oropharynx while axial magnets do not.

Alternatively, the internal magnetic member may comprise a material that is attractive to a magnet, with the proviso that the external magnetic member be a magnet. In other embodiments, the internal magnetic member may comprise a radiopaque material. In some embodiments, all or a portion of the internal magnetic member may be covered by a coating, non-limiting examples of which include plastics, including but not limited to, polypropylene, polybutadiene, polvinyl chloride, biopolymers, thermoplastic polymers, and combinations thereof.

The internal magnetic member 32 may be disposed at any appropriate position substantially near the distal end 22 of the wire 18. As used herein, “substantially near” refers to a distance from the distal end of the wire 18 of less than about 10%, less than about 5%, less than about 2.5%, or less than about 1% of the length of the entire wire 18.

In some embodiments, the internal magnetic member 32 may be integrally formed in the wire 18. For example, the internal magnetic member 32 may be partially embedded in a portion of the distal end 22 of the wire 18 or partially enclosed within the distal end 22 of the wire 18 such that the internal magnetic member 32 is at least partially encapsulated by the wire 18. In some embodiments, a portion of the internal magnetic member 32 may be encapsulated substantially near the distal end 22 of the wire 18 while the remaining portion of the internal magnetic member 32 extends beyond the distal end 22 of the wire 18. In such embodiments, the portion of the internal magnetic member 32 that is not otherwise encapsulated within the wire 18 optionally may be covered with an internal magnet coating. In still other embodiments, the entire internal magnetic member 32 may be fully encapsulated within wire 18. In other embodiments, the internal magnetic member 32 may be fixed at the distal end 22 of the wire 18. In such embodiments, the internal magnetic member 32 optionally may extend beyond the distal end 22 of the wire 18 by a distance of less than about 10%, less than about 5%, less than about 2.5%, or less than about 1% of the entire length of the wire 18.

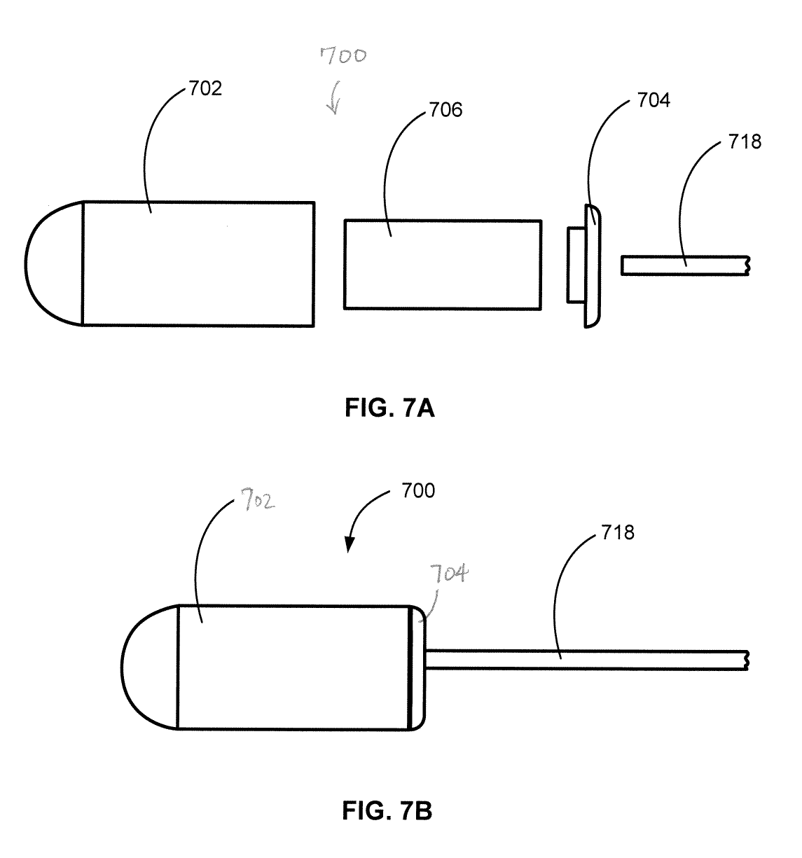

FIGS. 7A and 7B show a preferred embodiment of the internal magnet member 700, which comprises a cylindrical magnet 706, which is housed in an encapsulator, which consists of an encapsulator body 702 and an encapsulator end cap 704. After the magnet 706 is inserted into a hollow space in the encapsulator body 702, the encapsulator end cap 704 is fixed to the encapsulator body 702 by any suitable means. For example, the encapsulator end cap 704 may be attached with an adhesive substance or by use of mechanical connector. The encapsulator end cap 704 is also connected to distal end portion of wire 718. The encapsulator body 702 and end cap 704 may be made of a non-magnetic material, such as aluminum, or a polymeric material, such as polyvinyl chloride. The distal tip of the encapsulator body should have smooth, rounded edges in order to navigate smoothly without injury to the patient.

In embodiments, the wire 18 may be configured to have a length such that a portion of the distal end 22 extends beyond the distal end 16 of the endotracheal tube 12. For example, in embodiments the distal end 22 of the wire 18 may extend about 0.25%, about 0.50%, about 0.75%, about 1%, or about 2% of the length of the wire 18 beyond the distal end 16 of the endotracheal tube 12. In other embodiments, the distal end 22 of the wire 18 may extend beyond the distal end 16 of the endotracheal tube 12 when the wire 18 is not fully inserted into the endotracheal tube 12.

In still other embodiments, the wire 18 is configured to have a length such that all or a portion of the internal magnetic member 32 extends beyond the distal end 16 of the endotracheal tube 12. For example, in embodiments, 100% of the internal magnetic member may extend beyond the distal end 16 of the endotracheal tube 12. In other embodiments, at least about 60%, at least about 50%, at least about 40%, or at least about 30% of the length of the internal magnetic member 32 extends beyond the distal end 16 of the endotracheal tube 12. As used herein, the immediately preceding percentages refer to the length of the internal magnetic member extending beyond the distal end of the endotracheal tube as compared to the entire length of the internal magnetic member.

End Cap

As described above, the wire 18 desirably is secured at its proximal end 20 to an end cap 24. In an embodiment, as illustrated in FIG. 2, the end cap 224 may comprise a first end 225, configured to be removably attached to the proximal end 14 of the endotracheal tube, and a second closed end 232. For example, the first end 225 may comprise an annular opening 227 defined by an outer annular wall 230 of the end cap 224 and an inner annular wall 226 of the end cap 224. The inner annular wall 226 may define any structure suitable for securely mounting the proximal end 20 of the wire 18 to the end cap 224.

The end cap 224 may comprise any material capable of being suitable secured to the wire and removably attached to the endotracheal tube. For example, in an embodiment the end cap may comprise a plastic or thermoplastic polymer. In a particular embodiment, the end cap 224 may comprise polyether ether ketone (PEEK). The end cap 224 may be secured to the proximal end 20 of the wire 18 using any suitable means, non-limiting examples of which include adhesives and thermal bonding. In some embodiments, the end cap 224 may further comprise a plurality of external grooves 228 or other structural features (i.e., elastomeric coating, ribbing, etc.) capable of improving the grippability of the end cap 224.

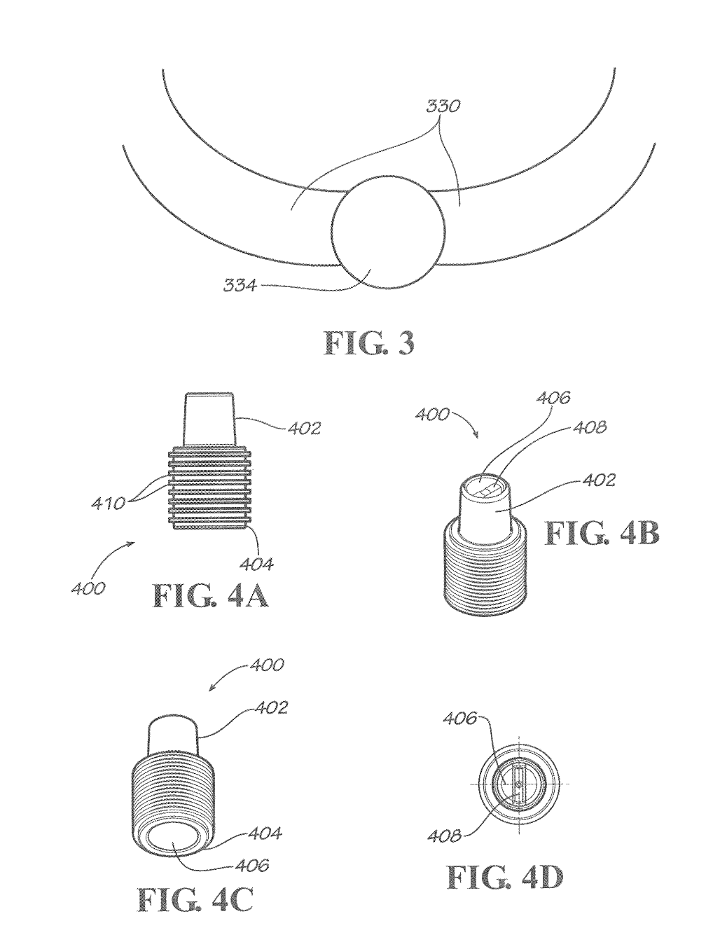

In other embodiments, the end cap may be configured to allow air to flow through the end cap. This embodiment is particularly suitable for use with a carbon dioxide monitor or detector for verifying proper intubation. For example, as illustrated in FIGS. 4A-4D, an end cap 400 comprises an open channel 406 that extends through the entire end cap 400 from a first end 402 of the end cap 400 to a second end 404 of the end cap 400. The first end 402 of the end cap 400 may have a bridge 408 or other suitable structure to connect to the end cap 402 to the wire (not shown). The wire may be secured to the end cap 400 such that it extends outwardly from the first end 402 of the end cap 400, such that the first end 402 is configured to be removably attached to the proximal end of the endotracheal tube or a respirator connector. As previously described, in embodiments the end cap 400 desirably comprises a plurality of grooves 410 or other suitable features to improve the ability of the medical professionals to grip the end cap and withdraw the wire and internal magnetic member from the endotracheal tube.

External Magnetic Member

The intubation system desirably further comprises an external magnetic member 34 to assist with guiding and positioning the endotracheal tube assembly 10 into the patient's trachea. The external magnetic member 334 may, for example, be removably attached to a harness 330 as illustrated in FIG. 3, to secure the external magnetic member at an appropriate position outside of the patient's body, for example, adjacent to the patient's cricothyroid cartilage. However, in other embodiments, the harness 330 is not used, such as when manual manipulation of the external magnetic member 334 is desired.

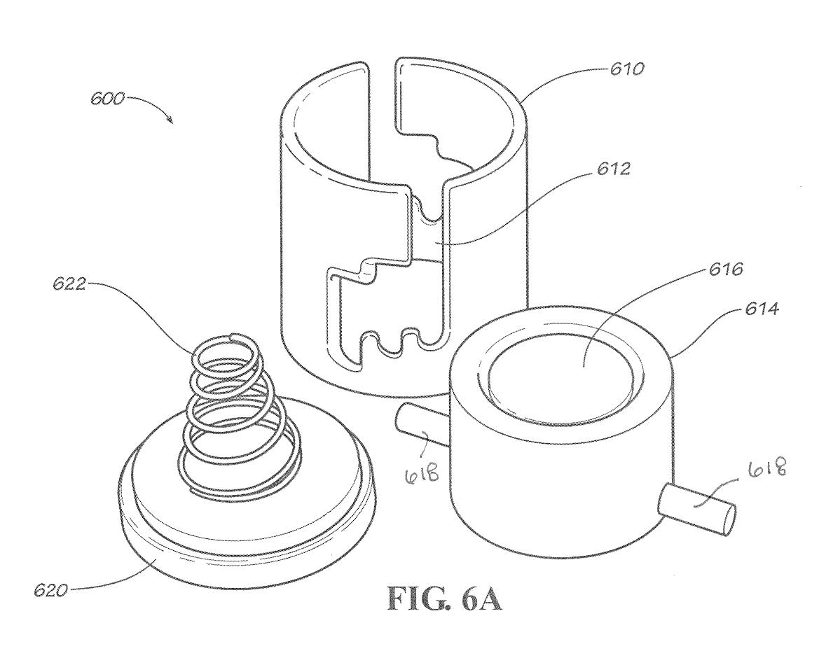

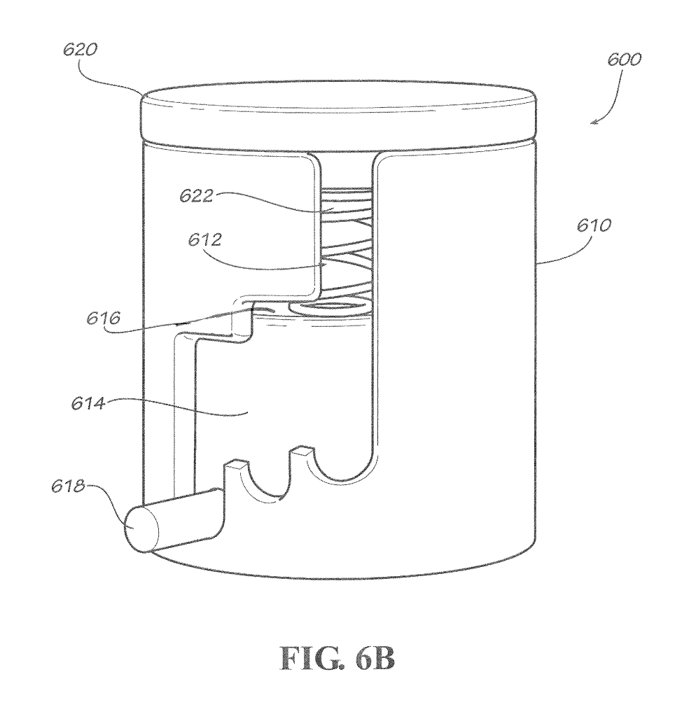

In one embodiment, illustrated in FIGS. 6A and 6B, the external magnetic member 600 comprises an outer shell 610 having one or more stair-stepped or similarly-shaped openings 612. Although a cylindrical shell is shown, numerous other shapes may be used (e.g., a quadrilateral shell, etc.). A base member 614 provides an inset in which at least one magnet 616 is inserted. The base member 614 further includes one or more levers 618 that extend through the openings 612 of the outer shell 610 when assembled. A top member 620 has a spring 622 extending therefrom. When assembled, the base member 614 and the top member 620 of the external magnetic member 600 are disposed in the outer shell 610 such that the one or more levers 618 of the base member 614 extend through the one or more openings 612 and the magnetic member 616 is disposed between the base member 614 and the spring 622 extending from the top member 620. In operation, the relative distance between the magnet 616 and the patient advantageously may be adjusted by adjusting the position of the base member 610 in the external magnetic member 600. That is, the different step positions of the levers enable the magnet to be disposed at different fixed distances between the bottom surface of the outer shell (which is placed against the patient) and the internal magnet member.

As used herein, the term “external magnetic member” means that the magnetic member is configured for use outside of a patient. In some embodiments, the external magnetic member may comprise a magnetic material, a ferromagnetic material, or a rare-earth magnet. In yet other embodiments, the external magnetic member may comprise a device that is capable of electrically and/or mechanically generating a magnetic field. In still other embodiments the external magnetic member may comprise a material that is attractive to a magnet, with the proviso that the internal magnet is a magnet.

The internal magnetic member and external magnetic member are of opposite polarity to each other so that movement of the external magnetic member will cause corresponding movement of the internal magnetic member. Thus, movement of the external magnetic member will allow one skilled in the art to properly position the endotracheal tube assembly guided by the internal magnetic member.

Method of Intubating a Patient

Method for intubating a patient are also provided. The method generally comprises positioning the external magnetic member adjacent to the cricothyroid cartilage of the patient. The patient may be a human adult or child. The endotracheal tube assembly 110 is either assembled and configured for deployment or may be provided already configured for deployment, as illustrated in FIG. 5. The endotracheal tube assembly 110 may be assembled, for example, by inserting the distal end 122 of the wire 118 into the proximal end 114 of the endotracheal tube 112 and using the end cap 124 to position and secure the wire 118 within the lumen of endotracheal tube 112. The endotracheal tube assembly 110 then may be inserted and guided into position in the trachea of the patient by the magnetic interaction between an external magnetic member and internal magnetic member 132 disposed at the distal end 122 of the wire 118. Desirably, the external magnetic member is positioned adjacent to the cricothyroid cartilage of the patient.

As described hereinabove, the movement of the external magnetic member allows one skilled in the art to properly position the endotracheal tube 112 which is guided by the internal magnetic member 132 disposed at a distal end 122 of the wire 114. In embodiments, the internal magnetic member 132 may be disposed substantially near the distal end 116 of the endotracheal tube 112, or beyond the distal end 116 of the endotracheal tube 112, when the wire 118 is fully inserted into the endotracheal tube 112 and the end cap 124 is removably attached to the proximal end 114 of the endotracheal tube 112 via respirator connector 136.

In some embodiments, tactile feedback on the external magnetic member may be used to confirm appropriate positioning of the endotracheal tube 112. Alternatively, a carbon dioxide detector disposed in the end cap, as illustrated in FIG. 4 for example, may be used to detect whether the intubation has been successful (e.g., by using a means for detecting carbon dioxide flow to detect the flow of carbon dioxide, non-limiting examples of which include papers that change color from purple to yellow when carbon dioxide flows through the through-channel).

After it has been determined that the intubation has been successful and the endotracheal tube 112 is properly positioned in the trachea of the patient, the wire 118 is removed from the patient and/or the endotracheal tube assembly 110 by gripping the end cap 124 about grooved surface 128 and withdrawing the wire 118, along with the internal magnetic member 132, from the endotracheal tube 112. The external magnetic member also is removed from the patient, either before or after withdrawal of the wire 118.

Additional aspects and features of the endotracheal tube assembly 110 that may be used in the method for intubating a patient are similar to those aspects and features described hereinabove for the endotracheal tube intubation system.

The present invention may be further understood with reference to the following non-limiting examples.

### Example 1

### First Working Embodiment

An endotracheal tube intubation system was fabricated, assembled, and tested with in a model trachea assembly. The configuration having the following specifications was found to be effective at properly positioning an endotracheal tube within the model trachea assembly:

Internal magnetic members: 4 inline rare-earth magnetic spheres; diameter: 0.25 inches each; pull force: 4.9 pounds of pull for each sphere; length of the 4 inline spheres: 1.0 inch.

External magnetic member: 5 stacked rare-earth magnetic disks; total height: 0.25 inches; diameter: 1.5 inches; pull force: 65 pounds of pull for each disk.

Endotracheal tube: inner diameter: 0.50 inches; length: 13.25 inches.

Resuscitator connector: material: PVC; outer diameter: 0.50 inches.

End cap: material: PEEK; diameter: 1 inch; length: 1 inch.

Flexible wire: material: PVC; diameter: 0.125 inches; length: 14.5 inches.

### Example 2

### Second Working Embodiment

An endotracheal tube intubation system was fabricated, assembled, and tested with in a model trachea assembly. The configuration having the following specifications was found to be effective at properly positioning an endotracheal tube within the model trachea assembly:

Internal magnetic members: 4 inline rare-earth magnetic spheres; diameter: 0.25 inches each; pull force: 4.9 pounds of pull for each sphere; length of the 4 inline spheres: 1.0 inch.

External magnetic member: 1 rare-earth magnetic disk; diameter: 2.0 inches; pull force: 90 pounds of pull.

Endotracheal tube: inner diameter: 0.50 inches; length: 13.25 inches.

Resuscitator connector: material: PVC; outer diameter: 0.50 inches.

End cap: material: PEEK; diameter: 1 inch; length: 1 inch.

Flexible wire: material: PVC; diameter: 0.125 inches; length: 14.5 inches.

### Example 3

### Third Working Embodiment

An endotracheal tube intubation system was fabricated, assembled, and tested with in a model trachea assembly. The configuration was similar to the first two examples; however, the internal magnetic member included a single cylindrical neodymium magnet (N52 caliber) approximately 4 mm diameter and 16 mm length, housed in an aluminum housing having a wall thickness of about 1 mm with an overall length of about 24 mm. The housing has rounded edges. In addition, the injection molded end cap was modified to include a through-channel and a commercially-available carbon dioxide monitor (changes color from purple to yellow when exposed to a high concentration of carbon dioxide, only afforded when in the tube is placed in the trachea.

It should be apparent that the foregoing relates only to the preferred embodiments of the present invention and that numerous changes and modifications may be made herein without departing from the spirit and the scope of the invention as defined by the following claims and equivalents thereof.

We claim:

1. An endotracheal intubation system comprising:
an endotracheal tube assembly which comprises:
an endotracheal tube having a proximal end and a distal end;
a wire having a proximal end and a distal end, wherein the wire is configured to be removably inserted into the endotracheal tube through the proximal end of the endotracheal tube;
an internal magnetic member fixed at the distal end of the wire; and
an end cap attached to the proximal end of the wire and removably securable to the proximal end of the endotracheal tube, wherein at least a portion of the internal magnetic member is disposed within the distal end of the endotracheal tube at a fixed position when the end cap is secured to the proximal end of the endotracheal tube; and
an external magnetic member positionable on the exterior of a patient.

2. The intubation system of claim 1, wherein at least a portion of the internal magnetic member extends beyond the distal end of the endotracheal tube when the end cap is secured to the proximal end of the endotracheal tube.

3. The intubation system of claim 2, wherein about 50% of the length of the internal magnetic member extends beyond the distal end of the endotracheal tube.

4. The intubation system of claim 1, wherein the end cap is removably connected to the proximal end of the endotracheal tube via a resuscitator connector connected therebetween.

5. The intubation system of claim 4, wherein the end cap comprises a first end configured to removably attach to a proximal end of the resuscitator connector.

6. The intubation system of claim 5, wherein the first end comprises an annular opening defined by an outer annular wall and an inner annular wall, and wherein the proximal end of the resuscitator connector mates by frictional engagement within the annular opening.

7. The intubation system of claim 1, wherein the internal magnetic member comprises a plurality of rare-earth magnetic spheres aligned within an cylindrical housing.

8. The intubation system of claim 1, wherein the cylindrical housing has a length of about 1 inch and a diameter of about 0.25 inches.

9. The intubation system of claim 1, wherein the end cap further comprises a plurality of external ridges or grooves.

10. The intubation system of claim 1, wherein the internal magnetic member and the external magnetic member are of opposing polarities.

11. The intubation system of claim 1, wherein the end cap further comprises a carbon dioxide detector.

12. The intubation system of claim 1, wherein the external magnetic member comprises an outer shell, a magnet disposed therein, and an adjustable positioning member for selectively securing the magnet in two or more positions within the outer shell.

13. A kit of parts comprising:
a first part comprising a wire having a proximal end and a distal end, an internal magnetic member fixed at the distal end of the wire, and an end cap attached to the proximal end of the wire, wherein the end cap is removably securable to a proximal end of an endotracheal tube with the wire removably inserted into the endotracheal tube and at least a portion of the internal magnetic member disposed within the distal end of the endotracheal tube; and
a second part comprising external magnetic member positionable on the exterior of a patient,
wherein the first and second parts are configured to cooperate to magnetically guide the endotracheal tube into a patient's trachea.

14. The kit of claim 13, further comprising at least one endotracheal tube.

15. The kit of claim 14, wherein the external magnetic member comprises an outer shell, a magnet disposed therein, and an adjustable positioning member for selectively securing the magnet in two or more positions within the outer shell.

16. A method for intubating a patient comprising:
positioning an external magnetic member adjacent to a cricothyroid cartilage of the patient;
inserting though the patient's mouth and into the patient's trachea a distal end of an endotracheal tube assembly which comprises (a) an endotracheal tube comprising a flexible, hollow tube having a distal end and a proximal end, (b) a wire having a proximal end and a distal end, (c) an internal magnetic member fixed at the distal end of the wire, and (d) an end cap fixed to the proximal end of the wire and removably secured to the proximal end of the endotracheal tube, wherein at least a portion of the internal magnetic member is disposed within the distal end of the endotracheal tube; and then
unsecuring the end cap from the proximal end of the endotracheal tube and withdrawing the wire and internal magnetic member from the endotracheal tube through the proximal end of the endotracheal tube, while maintaining the endotracheal tube within the patient's trachea.

17. The method of claim 16, wherein the internal magnetic member is positioned adjacent to the cricothyroid cartilage of the patient using the external magnetic member.

18. The method of claim 16, further comprising confirming intubation of the patient using a carbon dioxide detector before withdrawing the wire and internal magnetic member from the endotracheal tube.

19. The method of claim 16, further comprising adjusting attraction of the internal magnetic member to the external magnetic member by manually adjusting a distance between the external magnetic member and the internal magnetic member.
